(19) United States
(12) Patent Application Publication
(10) Pub. No.: US 2013/0085355 A1
(43) Pub. Date: Apr. 4, 2013

(54) MONITORING SYSTEM

(71) Applicant: University of Limerick, Plassey Technological Park (IE)

(72) Inventors: Ulrich Timm, Kritzmow (DE); Jens Kraitl, Schwerin (DE); Sebastian Koball, Rostock (DE); Michael V. Hinz, Rostock-Elmenhorst/Lichtenhagen (DE); Sergej Andruschenko, Rostock (DE); Hartmut Ewald, Rostock (DE); Elfeld Lewis, County Clare (IE); Deirdre Mcgrath, County Limerick (IE); Gabriel Leen, County Cork (IE)

(73) Assignee: UNIVERSITY OF LIMERICK, Limerick (IE)

(21) Appl. No.: 13/629,766

(22) Filed: Sep. 28, 2012

Related U.S. Application Data
(60) Provisional application No. 61/541,389, filed on Sep. 30, 2011.

(51) Int. Cl. A61B 5/02; A61B 5/1455

(52) U.S. Cl. 600/324; 600/479

(57) ABSTRACT

A system and method for non-invasive monitoring of cardiac activity in a human or animal is disclosed. A radiation source directs radiation through a patient site, and a detector detects radiation after passing through a patient tissue. A processor processes data derived from the detected radiation, determining pulse peaks and troughs and calculating area under a pulse peak to provide a real time cardiac output indicator. The radiation wavelength is on a haemoglobin spectral isosbestic point, not influenced by changes in SpO2 concentration. The processor performs numerical integration of pulse data between troughs, and wherein said integration is performed per pulse. Preferably, the processor monitors trends, thus providing very useful information and reducing need for calibration.

### FIELD OF THE INVENTION

The invention relates to monitoring cardiac activity of a patient.

### PRIOR ART DISCUSSION

Several semi- or non-invasive cardiac output (CO) tracking techniques considered to be a current state-of-the-art have some positionally significant limitations and ambiguities. Some of them require not only an arterial catheterization, but the additional operator inputs of the patient-specific characteristics (e.g. gender, age, weight), features of the pressure waveform (e.g. skewness, kurtosis). Some of these techniques are also based on the numeral assumptions (e.g. constant cross-sectional area of the vessels over time, ideal geometry of the anatomic structures, flat flow velocity profile, etc). These limitations are especially relevant in the case of monitoring cardiac function.

It is also known to provide a non-invasive pulse spectroscopy system for real time monitoring of cardiac activity. The following references relate to the field of non-invasive blood monitoring using optical emitters and detectors.
[1] Kraitl J et al: “Optical sensor technology for a non-invasive continuous monitoring of blood components”, Optical Diagnostics and Sensing X: Toward Point-of-care Diagnostics 25-26 Jan. 2010 San Francisco, Calif., USA, vol. 7572, 25 Feb. 2010 (2010 Feb. 25).
[2] Timm U et al: “Optical sensor system for non-invasive blood diagnosis”, Sensors Applications Symposium, 2009, SAS 2009, IEEE, IEEE, Piscataway, N.J., USA, 17 Feb. 2009 (2009 Feb. 17) pages 240-244.
[3] Timm U et al: “Non-invasive continuous online haemoglobin monitoring system”, Sensors Applications Symposium (SAS), 2010 IEEE, IEEE, Piscataway, N.J., USA, 23 Feb. 2010 (2010 Feb. 23) pages 131-134.
[4] Timm U et al: “Non-invasive optical real-time measurement of total haemoglobin content”, Procedia Engineering, vol. 5, 1 Jan. 2010 (2010 Jan. 1) pages 488-491
[5] Timm U et al: “Sensor system for non-invasive optical haemoglobin determination”, Sensors, 2009 IEEE, IEEE, Piscataway, N.J., USA, 25 Oct. 2009 (2009 Oct. 25), pages 1975-1978.

Also, U.S. Pat. No. 6,325,762 describes a system for cardiac output monitoring, in which resistance within the blood vessel is measured. US2011172518 describes an approach in which a permanent magnet arrangement is used for non-invasive measurement of cardiac output. US2010222658 describes an approach in which reflection of light of different wavelengths from external tissue is measured. US2010152591 describes an approach in which an acoustic energy transducer provides sensing data.

The invention is directed towards achieving improved cardiac activity monitoring.

### SUMMARY OF THE INVENTION

According to the invention, there is provided a system for non-invasive monitoring of cardiac activity in a human or animal, the system comprising:
a radiation source,
a driver for the radiation source,
a radiation detector, and
a processor for processing data derived from the detected radiation, in which the processor is adapted to determine pulse peaks and troughs and to calculate area under a pulse peak to provide a real time cardiac output indicator data.

In one embodiment, the processor is adapted to perform tracking of a cardiac output trend in addition to or instead of an absolute cardiac output value.

In one embodiment, the radiation source and the detector are arranged to operate on either the transmissive or the reflectance principles.

In one embodiment, the system is adapted to acquire a non-invasive signal by irradiating a measuring site with the radiation source operating at a wavelength on a haemoglobin spectral isosbestic point, in which radiation is modulated thereafter by blood circulation activity.

In one embodiment, the processor is adapted to perform numerical integration of pulse data between troughs, and wherein said integration is performed per pulse.

In one embodiment, the processor is adapted to convert said indicator data to cardiac output data by usage of a predefined calibration curve based on cardiac output values from a large patient pool.

In one embodiment, the processor is adapted to perform numerical integration of pulse data between troughs, and wherein said integration is performed per pulse; and wherein the processor is adapted to integrate by executing an adaptive function.

In one embodiment, the processor is adapted to calculate cardiac output values correlating with beat volume units based on the terms of a predetermined empirical calibration curve derived from thermodilution measurements of cardiac output.

In one embodiment, the processor is adapted to calculate cardiac output values based on a predetermined empirical calibration curve derived from thermodilution measurements of cardiac output, to determine beat volume units based on stroke index, and to combine the beat volume units with cardiovascular characteristics of a patient.

In one embodiment, the processor is adapted to determine cardiac output measurement data and to calibrate said data using a thermodilution technique. Preferably, the processor is adapted to execute an autoregulatory compensation algorithm.

In one embodiment, the system comprises a plurality of pairs of radiation sources and detectors, and the processor is adapted to process data from said plurality of detectors.

In one embodiment, the system comprises a plurality of pairs of radiation sources and detectors, and the processor is adapted to process data from said plurality of detectors and said sensors, and the system further comprises at least one non-optical sensor; and said non-optical sensor includes at least one ECG sensor.

In one embodiment, the processor is adapted to estimate haemoglobin content and blood oxygen concentration, and to derive from said estimations an estimate of total oxygen uptake.

In another aspect, the invention provides a method for non-invasive monitoring of cardiac activity in a human or animal, the method comprising:
a radiation source directing radiation through a patient site,
a detector detecting radiation after passing through a patient tissue, and
a processor processing data derived from the detected radiation, in which the processor determines pulse peaks and troughs and calculates area under a pulse peak to provide a real time cardiac output indicator.

In one embodiment, the processor performs tracking of a cardiac output trend in addition to or instead of an absolute cardiac output value.

In one embodiment, the radiation source emits radiation with wavelength at haemoglobin or SpO2 spectral isosbestic points, in which radiation is modulated thereafter only by blood circulation activity.

In one embodiment, the processor performs numerical integration of pulse data between troughs, and wherein said integration is performed per pulse.

In one embodiment, the processor converts indicator data to beat volume units.

In one embodiment, the processor performs numerical integration of pulse data between troughs; and wherein the processor integrates by executing an adaptive function.

In one embodiment, the processor calculates cardiac output values correlating with beat volume units based on the terms of a predetermined empirical calibration curve derived from thermodilution measurements of cardiac output.

In one embodiment, the processor calculates cardiac output values based upon an equation combining beat volume units with individual cardiovascular characteristics of a patient.

In one embodiment, the radiation source and the detector are applied at a peripheral patient location.

In one embodiment, the processor executes an autoregulatory compensation algorithm.

In a further aspect, the invention provides a computer readable medium comprising software code adapted to perform, when executed by a digital processor, a method comprising the steps of:
receiving data from a detector which detects radiation after passing through a patient tissue, and
processing data derived from the detected radiation, to determine pulse peaks and troughs and to calculate area under a pulse peak to provide a real time cardiac output indicator.

### DETAILED DESCRIPTION OF THE INVENTION

### BRIEF DESCRIPTION OF THE DRAWINGS

The invention will be more clearly understood from the following description of some embodiments thereof, given by way of example only with reference to the accompanying drawings in which:

FIG. 1 is a high-level diagram illustrating a cardiac activity monitoring system of the invention, based on the transmissive principle;

FIG. 2 is a flow diagram for operation of the system of FIG. 1;

FIG. 3 illustrates graphically the nature of blood flow detection with the system of FIG. 1;

FIG. 4 is a set of plots, showing sensed amplitude, its first derivative, and its second derivative;

FIG. 5 is a plot showing showing how the sensed optical data is processed to generate cardiac output data;

FIG. 6 is a flow diagram illustrating operation of a processor using the data of the plots of FIGS. 4 and 5;

FIG. 7 is an example flow diagram for processing the detected light (PPG) signal;

FIG. 8 is a diagram illustrating the principle of operation of a reflectance probe of a system of an alternative embodiment; and

FIG. 9 is a diagram illustrating the arrangement of an alternative embodiment, in which several sensors are utilised to achieve a more accurate measurement

### DESCRIPTION OF THE EMBODIMENTS

Referring initially to FIGS. 1 to 3, a monitoring system 1 of the invention has a processor 2, light emitting diode (LED) drivers 3, LEDs 4, and photodiodes (PDs) 5. It operates non-invasively by directing transmitting radiation through a patient's body at a selected wavelength, preferably an isosbestic wavelength of 810 nm or 1310 nm or at other frequencies which are higher than 1300 nm, as most wavelengths greater than 1300 nm are Hb-isosbestic.

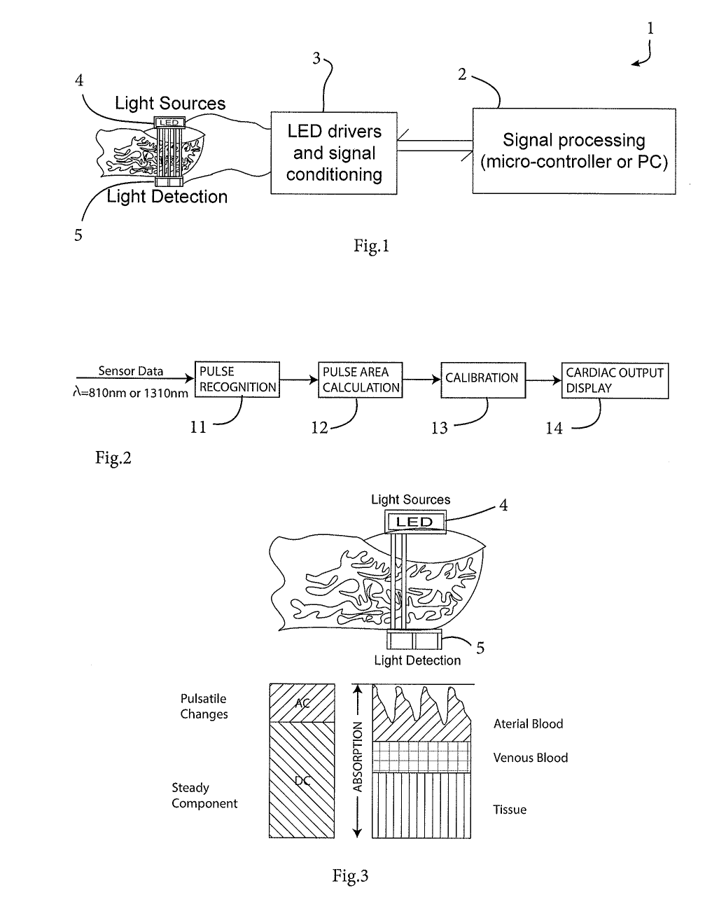

As shown in FIG. 2, the main steps are pulse recognition 11, pulse area calculation 12, calibration 13, and provision of a cardiac output display 14. The radiation sensed by the PD 5 is modulated by blood circulation activity, with a high signal-to-noise ratio. This is illustrated in FIG. 3, in which can be seen n AC component and a DC component.

In more detail, the sensed radiation pulses are recognized by the processor. Importantly, the pulse area is calculated. This provides an integration of the pulse. After calibration, an output of cardiac activity is generated.

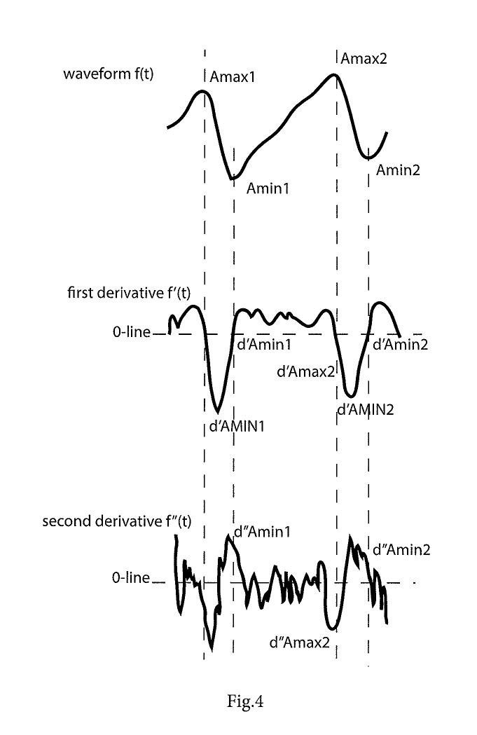

As shown in FIG. 4, the amplitude of f(t) corresponds to the signal output from the PD 5 in volts. The subsequently differentiated signals f′(t) and f″(t), are the first and second derivatives of f(t), with respect to time. The first derivative f′(t) may be interpreted as the rate of change of the signal f(t). The second derivative f″(t) may be interpreted as a measure of how the rate of change of the signal f(t) is itself changing or it may be understood as the rate of acceleration of the signal f(t).

The maxima and minima of the f(t) plot are represented by the zero values in the f′(t) plot, where the first derivative function f′(t) closes the zero-line. These zero values provide boundaries for the processor to calculate the area under the f(t) curve, as shown in FIG. 5. The second derivative shown at the bottom of FIG. 4 provides additional useful information for the data processing, such as that related to criteria for local minimum or local maximum of the function. Falsely-detected local extrema can be eliminated by thresholding the extrema's absciss points within a temporal sliding window. For example, if there is a series of at least two minima in succession due to a small dip in f(t) following the major dip, then the major dip within the temporal sliding window will be detected as the local minimum. Often it is the case that during processing of the PD signal some distinct dicrotic notches can falsely appear as local extrema and hence techniques such as utilising the second derivative need to be employed to deal with such occurances.

A transmission measuring principle is used to transmit light at different frequencies through the subject's tissues and thereafter to determine the pulsatile and non-pulsatile components of the received light at the sensor site (see FIG. 3). The method of optical transmission differs from optical reflectance by virtue of the location of light sources and detector, with respect to each other, which are placed on opposite sides of the investigated tissues, and transmit the light through the investigative tissue. The received optical signal is a waveform, which results from the effects of the systolic and diastolic contraction phases of the heart on the blood vessels, and the superposition of the light absorbed by all the biological tissues located on the optical path. Mathematically it is described by the Beer-Lambert's law, which for more than one absorbing substance present is:

$${A}_{t} = {\sum }_{i = i}^{n} {e}_{i} ( λ ) {c}_{i} {d}_{i}$$

where Aₜ is a total absorbance of a medium with n absorbing substances, e, c and d are respectively the extinction coefficient, molar concentration of absorbing species in the material, and optical path length of the ambient, λ—wavelength.

In order to compensate for the changes in a radiation source intensity, every pulse square S₁(AC) is normalized with the square of non-pulsatile part S₂(DC) giving a so called stroke index (SI):

SI=S₁(AC)/S₂(DC)

A value of the SI is then used by the algorithm as a key element in cardiac output estimation. S₁(AC) represent a pulsative blood volume and S₂(DC)—constant non-pulsative blood volume (e.g. soft tissues, bones). SI correlates with a total blood volume being circulated in the body.

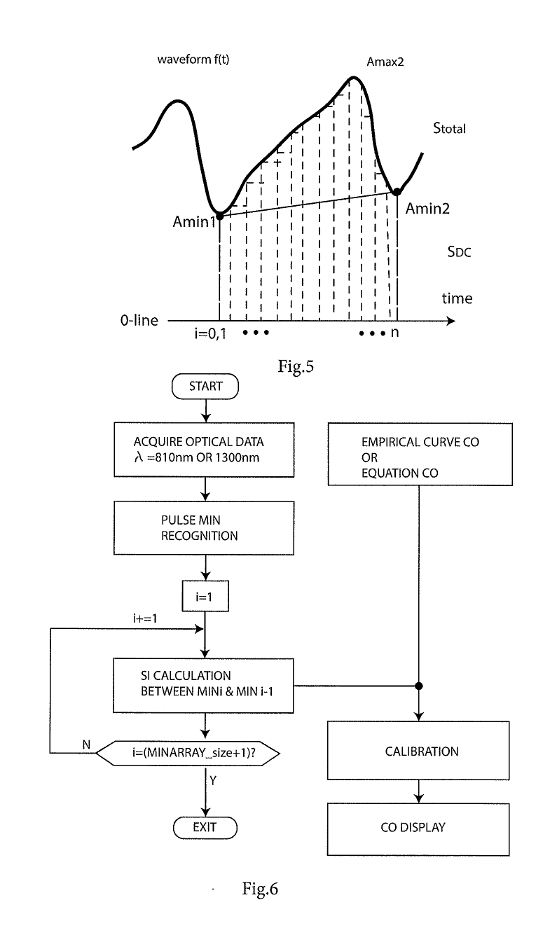

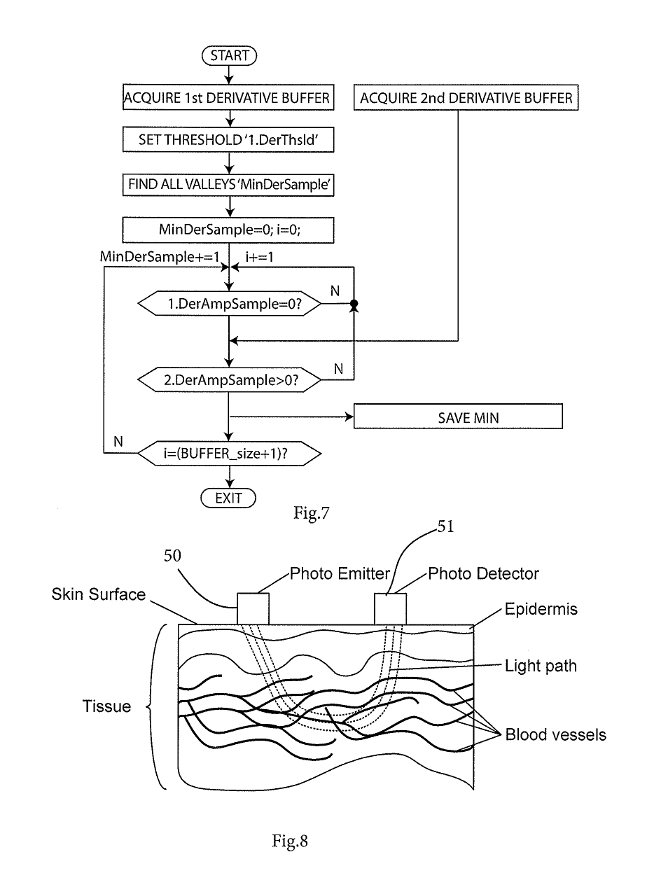

FIG. 6 provides an example flowchart of the software executed by the data processor. To separate the single pulses initially the individual peaks and valleys are determined by the double-derivation scheme. FIG. 7 provides an example implementation of the double-derivation scheme for pulse recognition. In this particular case, a derivation method of the 4th order is given by the following equation:

$${f}^{′} ( {t}_{i} ) = \frac{1}{12 dt} ( - {x}_{i + 2} + 8 {x}_{i + 1} - 8 {x}_{i - 1} + {x}_{i - 2} )$$

for i=0, 1, 2, . . . , n-1,

where n is the number of samples in f(t).

In order to distinguish the troughs framing individual pulses, a threshold for the 1st derivative of the original data was set. Thus, the individual processing windows are determined, as in-between d′MIN1 and d′MIN2 on FIG. 4. Then the first sample d′A_{MIN1} of the 1st derivative buffer f′(t) which crosses a zero-line is detected. After processing the 2nd derivative buffer f″(t), where d″A_{MIN1}>0, the point A_{MIN1} is identified as the first local minimum within the pulse. Correspondingly, sample d′A_{MIN2} of the 1st derivative buffer f′(t) which crosses a zero-line is detected and d″A_{MIN2}>0 is identified. Thus, the point A_{MIN2} is identified as the second local minimum within the same pulse. Both minima correspond to the end of the systolic phase and beginning of the diastolic phase. Point A_{MIN2} is identified as the pulse local maximum, which corresponds to the end of the diastolic phase and beginning of the systolic phase.

When peaks and valleys which frame every particular pulse are identified, the energy of every pulse can be calculated. The energy of the pulse is mathematically a square of the sample points under the curve and is described by:

$$E = {{\sum }_{i}}^{N} {q}_{i} Φ ( {r}_{i} )$$

where a group of N charges qᵢ at positions rᵢ is considered. For each i value, Φ(rᵢ) is the electrostatic potential due to all point charges.

It allows use of a complete pulse waveform information located in between two predetermined troughs, composing a single pulse. The method is an analogy of the calculation of electric potential energy E in electricity.

Calculation of an area under the pulse curve is based upon numerical integration using a quadrature approach, (in this particular implementation, however other methods may be used) i.e. summarising the integrated parts of the pulse. The sum of the integrals under the curve represent a total square or energy of the single pulse (converted later to beat volume units), as shown on FIG. 5. The pulse integral part may be numerically evaluated using for example an adaptive Gauss-Lobatto quadrature. Gauss-Lobatto quadrature is an approximation of the definite integral of a function, usually stated as a weighted sum of function values at specified points within the domain of integration. It is based on the following two rules: integration points include the end points of the integration interval; and it is accurate for polynomials up to degree 2n-3, where n is the number of integration points. If f(x) does not have many derivatives at all points, or if the derivatives become large, then Gaussian quadrature is often insufficient. For example, a Lobatto quadrature shown in (equation 2 below) calculates adaptively the definite integral of a function f(x) on interval [0, 1], by choosing smaller steps near the problematic points:

$${\int }_{0}^{1} f ( x ) d x = \frac{2}{n ( n - 1 )} \cdot [ f ( 0 ) + f ( 1 ) ] + {\sum }_{i = 2}^{n - 1} {w}_{i} \cdot f ( {x}_{i} ) + {R}_{n}$$

where xᵢ is the (i−1)-st zero of P′(x)ₙ₋₁; weights

$${w}_{i} = \frac{2}{{n ( n - 1 ) [ {P}_{n - 1} ( {x}_{i} ) ]}^{2}} ,$$

xᵢ≠0, 1; remainder

$${R}_{n} = \frac{- {n ( n - 1 )}^{3} \cdot {{2}^{2 n - 1} [ ( n - 2 ) ! ]}^{4}}{( 2 n - 1 ) \cdot {[ ( 2 n - 2 ) ! ]}^{3}} \cdot {f}^{( 2 n - 2 )} ( ξ ) , ( 0 < ξ < 1 ) .$$

There is a systematic error in estimation of area under the curve due to the quadrature method itself, but it influences the results minimally due to the equal algorithm-based calibration. This error due to the Gauss-Lobatto quadrature is typically less than 1%. However, other techniques which have lower error range may provide better accuracy (e.g. Gaussian or Gauss-Kronrod quadratures).

To calculate the area under the individual pulse by the quadrature-based method, the non-pulsative square S_{DC} is substracted from the total calculated square S_{TOTAL}.

The heart beat volume (HBV) and the heart rate (HR) determine the heart minute volume (HMV) or cardiac output (CO) as an important value for circulatory regulation:

HMV=HR·HBV

The HR value can be determined using the same optical sensor by processing of the pulse plethysmographic waveform either in frequency or in time domain dependent on the hardware specification to meet a continuous real-time operation mode. In temporal domain the pulse rate is usually extracted as quantity of the PPG-pulses per data buffer. The plethysmographic waveform can be converted to the frequency domain by the Fourier transform (or fast Fourier transform (FFT) technique). And the HR is identified as the fundamental frequency harmonic with the highest power energy in a power spectrum and subsequently converted to the beats/minute units.

Cardiac output can be monitored in two modes, relatively and absolute. In relative mode only the relative changes of the SI during the measuring period are considered. These outputs are specific to the current monitoring session and might be used for comparative purposes with another monitoring sessions only after taking into account as far as possible all the monitoring conditions and physiological events, if some present. The absolute values of the CO, i.e. the volume of blood being pumped by the heart in minute in L/min, may be estimated through matching of the SI values with terms of a predetermined empirical calibration curve which has been derived empirically from a large data set of monitored probands. Such a calibration curve might be obtained through measurements of the CO parameter using a thermodilution-technique. Thermodilution involves injecting a cold (<8° C.) or room-tempered (<24° C.) saline solution through a central, or peripheral, line into the proband's circulatory system. The blood volume pumped by a ventricle in a minute can then be calculated by analysing the downstream temperature changes using a modified Stewart-Hamilton equation. Another alternative method to estimate CO-values is to use complex equations, which combine the SI outputs with individual cardiovascular characteristics, such as impedance, compliance, status of the vessels etc. Another method to achieve an absolute CO measurement for a particular measurement session is to initially calibrate the current device against another device which provides an initial reference calibration value. Recalibration may be required at intervals in order to keep the current device within a specified error tolerance.

An advanced sensor clip design assuring a constant or minimal pressure is recommended. The aim of this is to reduce the influence of the clip pressure on the measurement due to compression of tissues and/or blood stasis due to shrinkage of the vessels. All limitations inherent in standard pulse oximetry are similarly applicable to this spectroscopic method.

In one embodiment it will be appreciated that the above pulse cardio spectroscopy is a non-invasive and painless technique. The method can be accordingly applied to a larger patient population with a risk for hemodynamic instability. No potentially harmful intravenous, venous or arterial injections are required, which might lead either to the organ malfunctions, or in other cases allergic reactions, e.g. anaphylaxis. The beat-to-beat basis of the method allows a real-time continuous monitoring assuring a prompt medical therapeutic response.

Referring to FIG. 8, a reflectance measuring principle is characterized from the design point of view by virtue of the location of light sources 50 and detector 51, being in this case on the same side relative to the investigated tissues. A detected reflectance waveform is the superposition of the light absorbed by the biological tissues, and blood which are located on the optical path between the emitter and receiver. The reflectance waveform may look morphologically quite different from the transmission waveform for the same measuring site, as the optical path is different.

Preferably, a highly vascularized tissue area close to the central circulations can be used (e.g. ear or neck region above carotid artery, for reflection mode). These locations on the human body provide good sites for assessment of blood flow related parameters. The blood flow to organs essential for sustaining life do not decline appreciably unless the arterial pressure falls below the autoregulatory range as a result of, for example, hypotension caused by hypovolemia or circulatory shock. In addition, physiological effects such as vasoconstriction, and vasodilatation of the vessels are minimized at main blood-carrying vessels such as the carotid artery. Thus, the measurement error due to the autoregulation which is prevalent in the case of the smaller vessels is minimized by nodal application of the optical sensor. Thus, locating the sensor in proximity to main blood-carrying vessels is advantageous to the peripheral monitoring mode.

The sensor may alternatively be applied at the periphery (e.g. finger), however in this case an autoregulatory compensation algorithm is preferred, in order to minimise the measurement error due to the potential effects of autoregulation. The autoregulation of the cardiovascular system results from the Frank-Starling mechanism, when the heart stroke volume responds to the contractility of the cardiac muscle. An effect of the autoregulation mechanism on the periphery of the body (e.g. upper extremities) might be a vascular dilatation or contraction of the peripheral vascular bed, which can have an influence on the cardiac output estimation at distal measuring site.

The autoregulatory compensation algorithm may include an estimation of the central parameters and dynamic mechanical properties of an individual vascular system. These are a central blood pressure, peripheral vascular status (elasticity of the vessels), or vasomotory regulation. The peripheral monitoring mode in comparison with central mode is more practical due to the possibility of usage of the transmission measuring principle and simplicity of application.

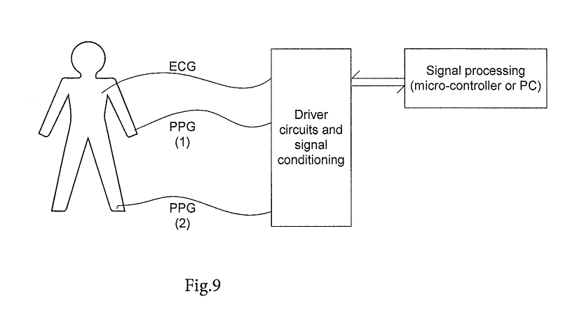

Referring to FIG. 9, a further embodiment of the invention includes a number of optical sensors (PPG(1) and PPG(2)) at different locations, for example a number of finger clips or a finger clip and a reflective probe on the carotid artery to provide multiple references to the cardiac output algorithm. Such a system may have improved accuracy and anomalies in the signal from one sensor can be compensated by referencing a second or third sensor, such as an Electrocardiography (ECG) input which provides data to the cardiac output algorithm and helps to improve the accuracy of the cardiac output measurement.

The system of the invention may be arranged to provide an estimation of blood oxygen saturation, heart rate, cardiac output and/or blood haemoglobin level and derive from these measurements an estimation of total oxygen uptake.

The invention is not limited to the embodiments described but may be varied in construction and detail.

1. A system for non-invasive monitoring of cardiac activity in a human or animal, the system comprising:
a radiation source,
a driver for the radiation source,
a radiation detector, and
a processor for processing data derived from the detected radiation, in which the processor is adapted to determine pulse peaks and troughs and to calculate area under a pulse peak to provide a real time cardiac output indicator data.

2. The system as claimed in claim 1, wherein the processor is adapted to perform tracking of a cardiac output trend in addition to or instead of an absolute cardiac output value.

3. The system as claimed in claim 1, wherein the radiation source and the detector are arranged to operate on either the transmissive or the reflectance principles.

4. The system as claimed in claim 1, wherein the system is adapted to acquire a non-invasive signal by irradiating a measuring site with the radiation source operating at a wavelength on a haemoglobin spectral isosbestic point, in which radiation is modulated thereafter by blood circulation activity.

5. The system as claimed in claim 1, wherein the processor is adapted to perform numerical integration of pulse data between troughs, and wherein said integration is performed per pulse.

6. The system as claimed in claim 1, wherein the processor is adapted to convert said indicator data to cardiac output data by usage of a predefined calibration curve based on cardiac output values from a large patient pool.

7. The system as claimed in claim 1, wherein the processor is adapted to perform numerical integration of pulse data between troughs, and wherein said integration is performed per pulse; and wherein the processor is adapted to integrate by executing an adaptive function.

8. The system as claimed in claim 1, wherein the processor is adapted to calculate cardiac output values correlating with beat volume units based on the terms of a predetermined empirical calibration curve derived from thermodilution measurements of cardiac output.

9. The system as claimed in claim 1, wherein the processor is adapted to calculate cardiac output values based on a predetermined empirical calibration curve derived from thermodilution measurements of cardiac output, to determine beat volume units based on stroke index, and to combine the beat volume units with cardiovascular characteristics of a patient.

10. The system as claimed in claim 1, where the processor is adapted to determine cardiac output measurement data and to calibrate said data using a thermodilution technique.

11. The system as claimed in claim 1, wherein the processor is adapted to execute an autoregulatory compensation algorithm.

12. The system as claimed in claim 1, comprising a plurality of pairs of radiation sources and detectors, and the processor is adapted to process data from said plurality of detectors.

13. The system as claimed in claim 1, comprising a plurality of pairs of radiation sources and detectors, and the processor is adapted to process data from said plurality of detectors and said sensors, and the system further comprises at least one non-optical sensor; and said non-optical sensor includes at least one ECG sensor.

14. The system as claimed in claim 1, wherein the processor is adapted to estimate haemoglobin content and blood oxygen concentration, and to derive from said estimations an estimate of total oxygen uptake.

15. A method for non-invasive monitoring of cardiac activity in a human or animal, the method comprising:
a radiation source directing radiation through a patient site,
a detector detecting radiation after passing through a patient tissue, and
a processor processing data derived from the detected radiation, in which the processor determines pulse peaks and troughs and calculates area under a pulse peak to provide a real time cardiac output indicator.

16. The method as claimed in claim 15, wherein the processor performs tracking of a cardiac output trend in addition to or instead of an absolute cardiac output value.

17. The method as claimed in claim 15, wherein the radiation source emits radiation with wavelength at haemoglobin or SpO2 spectral isosbestic points, in which radiation is modulated thereafter only by blood circulation activity.

18. The method as claimed in claim 15, wherein the processor performs numerical integration of pulse data between troughs, and wherein said integration is performed per pulse.

19. The method as claimed in claim 15, wherein the processor converts indicator data to beat volume units.

20. The method as claimed in either of claim 15, wherein the processor performs numerical integration of pulse data between troughs; and wherein the processor integrates by executing an adaptive function.

21. The method as claimed in claim 15, wherein the processor calculates cardiac output values correlating with beat volume units based on the terms of a predetermined empirical calibration curve derived from thermodilution measurements of cardiac output.

22. The method as claimed in claim 15, wherein the processor calculates cardiac output values based upon an equation combining beat volume units with individual cardiovascular characteristics of a patient.

23. The method as claimed in claim 15, wherein the radiation source and the detector are applied at a peripheral patient location.

24. The method as claimed in claim 15, wherein the processor executes an autoregulatory compensation algorithm.

25. A computer readable medium comprising software code adapted to perform, when executed by a digital processor, a method comprising the steps of:
receiving data from a detector which detects radiation after passing through a patient tissue, and
processing data derived from the detected radiation, to determine pulse peaks and troughs and to calculate area under a pulse peak to provide a real time cardiac output indicator.
